(19) United States
(12) Patent Application Publication
(10) Pub. No.: US 2009/0291123 A1
(43) Pub. Date: Nov. 26, 2009

(54) Opioid Combination Wafer

(76) Inventors: Hans-Rainer Hoffmann, Neuwied (DE); Reto Brändli, San Francisco, CA (US); Frank Theobald, Wehr (DE)

Correspondence Address: D. PETER HOCHBERG CO. L.P.A., 1940 EAST 6TH STREET, CLEVELAND, OH, 44114, US

(21) Appl. No.: 12/308,208

(22) PCT Filed: Jun. 4, 2007
(86) PCT No.: PCT/EP2007/004954

§ 371 Date: Jan. 22, 2009

(30) Foreign Application Priority Data
Jun. 16, 2006 (DE) 10 2006 027 793.7

(51) Int. Cl. A61K 9/70; A61P 25/24

(52) U.S. Cl. 424/443

(57) ABSTRACT

Sheet-like dosage forms for pain therapy, based on hydrophilic polymers, which rapidly dissolve or disintegrate in an aqueous environment and which release active agent combinations when placed into a body orifice or body cavity, and which are preferably orally administrable, with the dosage form containing an active agent combination consisting of an opioid and a second substance The second active agent is a non-steroidal anti-rheumatic (NSAR) or an antidepressant.

### CROSS-REFERENCE TO RELATED APPLICATIONS

This application is a National Stage application of International Application No. PCT/EP2007/004954, filed on Jun. 4, 2007, which claims priority of German application number 10 2006 027 793.7, filed on Jun. 16, 2006, both of which are incorporated herein by reference in their entireties.

### BACKGROUND OF THE INVENTION

1. Field of the Invention

The present invention relates to sheet-like dosage forms for pain therapy which rapidly dissolve or disintegrate in an aqueous environment and which release active agent combinations into a body orifice or body cavity, and which are preferably orally administrable, with at least one active agent being an opioid derivative.

2. Description of the Prior Art

Illnesses or injuries are usually accompanied by pain, with pain itself now being regarded as an illness and increasingly occurring as an independent symptom. In medical literature, pain is defined as an unpleasant sensory and emotional experience which is associated with injuries that have already occurred or are imminent, or which is perceived as such.

Pain can thus also occur without a clear cause, as an independent symptom (idiopathic pain). This form of idiopathic pain is not an exception but rather the rule. Thus, in 80% of all cases of back pain, for example, no pathological cause can be found, and the “invertebral disc”, for example, is responsible for the back pain in only 10% of all cases.

With “headaches”, too, so-called “primary headache” is the most frequent form. This means headache that cannot be attributed to an illness as a symptom thereof but which occurs without verifiable pathological change.

In addition, there are chronic pain disorders, with pain being defined as “chronic” if it lasts for more than 6 months and the causative affliction is either difficult to treat or untreatable, or if a cause for the pain cannot be found.

Chronic pain as a rule has a strong impact on the psyche of pain patients. It frequently leads to depression, and it strongly reduces the patient's quality of life.

Furthermore, research results have shown that pain conditions can be learned by the body. Repeatedly occurring pain leads to more intense and longer sensation of pain since frequent pain lowers the pain threshold. On the other hand, long-term administration of analgesics often does not show the permanent effect hoped for, and the treatment of the pain conditions frequently requires ever increasing dosages or more potent active agents to ensure that the patient is optimally treated.

In Germany, pain is often treated inadequately. This is particularly true of patients suffering from pain as a result of cancer, or in the case of postoperative pain. A reason for the above may lie in the restrictions imposed by the narcotics law and in the caution on the part of the physicians towards analgesics which fall under that law but which are extremely effective. Another cause may lie in the refusal of many patients to take a multitude of medicaments, certain groups of active agents or high dosages, linked with the fear of undesired drug effects (UDE) or dependence which are associated therewith, especially where opioid active agents are administered.

It has to be taken into consideration in this respect that in long-term therapy involving high dosages pathological damage and dependence do in fact occur, necessitating a discontinuance or change of medication and, as the case may be, withdrawal.

Apart from chronic pain, acute pain also requires treatment. Here, the focus is less on a long-term relief from pain. What is more important is achieving a quick onset of action and the associated alleviation of pain.

As already explained, pain constitutes a considerable impairment of the quality of life of the person afflicted.

### SUMMARY OF THE PRESENT INVENTION

It has therefore been the object of the present invention to provide a dosage form that can be utilised effectively in the therapy of pain and is quick-acting, but at the same time requires only a low dosage of active agents. In addition, the dosage form should have good compliance, that is, administration to the patient should be as simple as possible and the patient should not have reservations against taking the medication, for example on account of the size of the dosage form or the like. In addition, the disadvantages of the known dosage forms, particularly tablets, are to be avoided.

As mentioned above, acute pain conditions necessitate a quick, effective therapy. The dosage form should therefore be suitable for a quick release of active agent and for ensuring a quick onset of action. For this reason, the disintegration of the dosage form and the release of active agents should take place already at the site of application, in the case of orally administrable dosage forms, for example, already in the oral cavity.

In addition, it should be possible to apply the dosage forms in a simple and direct manner in order to facilitate intake also for patients with chronic pain.

Common dosage forms as used for administration of active agents in pain therapy are tablets, capsules, suppositories or injections.

Injections take effect quickly, but they are difficult to apply and they can practically not be used in public; the same applies to suppositories.

Tablets and capsules can be taken easily, but their onset of action is, as a rule, delayed, and the active agents, when being absorbed via the gastrointestinal tract, are subject to the “first-pass effect”, so that high initial concentrations of active agent in the tablets or capsules are required.

Moreover, as a rule some liquid is needed to swallow the dosage form, which is not always immediately available. Furthermore, the pain may make swallowing and chewing more difficult, so that even the application of effervescent tablets, sucking tablets or chewable tablets often turns out to be a problem.

Furthermore, it is known to use buccal or sublingual tablets that release the active agent in the oral cavity, so that the active agent can be absorbed directly via the oral mucous membrane.

The disadvantage of such tablets is an often unpleasant mouthfeel and, on account of the compact form, an only slow disintegration of the tablets, and, as a consequence thereof, a slow release of active agent.

Sheet-like wafer-like dosage forms, referred to as “wafers”, are known to be an alternative to the known buccal and sublingual tablets and to be a particularly advantageous dosage form for transmucosal administration of active agents.

To treat pain conditions, it is frequently necessary to use various active agents in order to ensure that the patient receives an optimal pain therapy. It is precisely this combining of active agents which calls for a strict intake according to an intake schedule to achieve the desired effect. For this reason, it is desirable to combine the possible active agents in one pharmaceutical form as this will make intake easier for the patient and minimise the risk of false applications.

It was found that the object is achieved by sheet-like administration forms made of a hydrophile polymer film that disintegrates in the oral cavity, these wafers containing at least two active agents, one of which is selected from the group of the opioids and a second one is selected from the group of the non-steroidal antirheumatics (NSAR's) or from the group of the antidepressants.

The combination of the active agents in the dosage form according to the invention makes it easier for the patient to take both active agents. The absorption of the active agents via the oral mucosa, as compared to other peroral dosage forms, affords, for example, the advantages that patients also having difficulty swallowing or patients refusing to take tablets can be administered medicaments via the oral route. In addition, the risk of medication errors is reduced since the patient has to take only one medicament for both of the active agents. Compliance and therapeutic success are thereby improved.

In addition, as a result of the direct absorption of the active agent via the mucous membrane, the time until the onset of action occurs is markedly reduced so that the patient perceives that the symptoms have abated within an extremely short time.

The fact that active agents are combined in one analgesic for pain therapy, with one active agent being an opioid, in particular, makes it possible to achieve special advantages. Thus, for example, a quick-acting, potent analgesic with a short half-life and a less quick-acting, less potent analgesic with a long half-life can be combined to give relief to a patient quickly and over a prolonged period of time.

Furthermore, a single active agent combination may contain active agents with different mechanisms of action having a synergistic effect, so that as a result of the different physiological activity in eliminating pain, lower amounts of the active agents can be dosed than would be the case with single-component compositions.

It is likewise possible to administer analgesic(s) and a further active agent, for example an antidepressant. This combination is often advantageous for persons suffering from chronic pain since, as already explained above, mental condition and enjoyment of life are often impaired in these persons. It was found that the sensitivity to pain is clearly higher in patients who are in a bad emotional state than in patients who are in a good frame of mind. Hence, the interaction of antidepressants for brightening the mood and analgesics likewise enables the use of lower dosages of the analgesic and thereby improved tolerance and low UDE.

Even where active agents are combined, a consistent intake and good compliance of the medicament are a prerequisite to ensuring optimal efficacy.

The administration of these active agent combinations in sheet-like dosage forms (wafers) not only enables easy intake but also the exact adaptation of the active agent components to each other, so that false dosages because intake has been forgotten or because of double intake of only one active agent, and thereby insufficient pain therapy, do not occur.

A further advantage of transmucosal administration of active agents consists in the circumvention of the gastrointestinal route and hence the avoidance of the “first-pass” effect after peroral administration, i.e. avoidance of the metabolism of a considerable portion of the active agent during the first liver passage, so that the active agent is exploited to a high degree.

Furthermore, direct absorption via the oral mucosa affords the advantage that even active agents which in long-term use or at high dosages irritate the gastric mucosa and/or which may lead to intolerance or even to severe UDEs, can be applied without these undesired effects.

In addition, loss of active agent due to the first-pass effect does not occur, so that the dosage of the active agents can be lowered correspondingly, which likewise leads to the patient being disburdened, and to improved well-being as a consequence of lower UDEs.

By varying the ratios of the active agents to each other, it is, in addition, possible to adapt the dosages to the respective needs. Thus, for example in the case of a chronic inflammatory illness, the active agent content of NSAR can be high, whereas the content of opioid for the alleviation of pain is kept as low as possible. On the other hand, the opioid content may be high while the NSAR content is lower, to alleviate acute, strong pain. Likewise, the opioid contents may correlate with the content of antidepressants, so that the focus may lie on the mood brightening effect, or on the pain treatment. If the patient's state of health changes, he or she can be easily stabilised on a dosage form with an altered active agent combination.

Because of the simple and low-cost manufacture of the wafers, it is possible to provide a large number of medicaments with different active agent concentrations.

Thus, if the wafer is made up of a laminate, it is possible, for example, to alter only the layer thickness of an active agent-containing layer, or to alter the concentration of the active agent.

On the other hand, medicaments can be produced which have different active agent contents but the same active agent ratio, simply by cutting the surface of the dosage form to different sizes.

Furthermore, because of their flat shape the wafers, containing active agent combinations, according to the invention can be carried along easily, e.g. in a wallet, and are available at once, even when travelling. They are easy to take and take effect quickly, both in chronic pain therapy and in the case of suddenly occurring attacks of pain, for example a migraine attack.

### DETAILED DESCRIPTION OF THE PRESENT INVENTION

Water-soluble or swellable polymers that are suitable as a base polymer for the hydrophilic water-soluble and/or swellable polymer film are polymers of the group comprising dextran, polysaccharides, inclusive of starch and starch derivatives, cellulose derivatives, such as carboxymethyl cellulose, ethyl cellulose or propyl cellulose, hydroxypropylmethyl cellulose, hydroxypropyl cellulose, sodium carboxymethyl cellulose (e.g. WALOCEL®), methyl cellulose, hydroxyethyl cellulose and hydroxypropylethyl cellulose, polyvinyl alcohols, polyethylene glycols, polyacrylic acids, polyacrylates, polyvinylpyrrolidones, alginates, pectins, gelatine, alginic acid, collagen, chitosan, arabinogalactan, galactomannan, agar-agar, agarose, carrageenan natural gums, tragacanth, highly dispersed silicon dioxide, bentonite, as well as derivatives of the aforementioned hydrophilic polymers, or combinations of two or more of these polymers. As an alternative, the polymer film may be made of a polyvinyl alcohol-polyethylene glycol graft copolymer.

The proportion of polymer in a dosage form according to the invention is preferably 5 to 95%-wt., more preferably 15 to 75%-wt., relative to the dry mass of the dosage form.

The active agent combinations according to the invention comprise at least one active agent from the group of the opioids and one active agent from the group of the NSAR's or the antidepressants, whereby it is also possible that at least one active agent from each one of these three active agent groups be contained in the active agent combination.

The active agent from the group of the opioids is selected from the group which comprises morphine, buprenorphine, hydromorphone, nalbuphine, fentanyl, sufentanil, alfentanil, remifentanil, tilidine, oxycodone, pethidine, levomethadone, piritramide, nalbuphine and pentazocine, as well as pharmacologically acceptable salts of these compounds, and combinations of two or more of the afore-mentioned compounds.

Preferably, morphine, hydromorphone, buprenorphine, oxycodone, nalbuphine and sufentanil are utilised as analgesic active agents.

The NSAR may be selected from the group which comprises acetylsalicylic acid, phenylbutazone, oxyphenbutazone, acemetacine, diclofenac, indomethacin, lonazolac, mefenamic acid, niflumic acid, ibuprofen, ketoprofen, naproxen, tiaprofenic acid, piroxicam, tenoxicam and meloxicam, as well as pharmacologically acceptable salts and combinations of these active agents.

Preferably, diclofenac, piroxicam, tenoxicam and meloxicam are utilised as analgesic and anti-inflammatory active agents.

The antidepressant may be selected from the group which comprises phenothiazines, azaphenothiazines, thioxanthenes, butyrophenones, diphenylbutyl piperidines, iminodibenzyl derivatives, iminostilbene derivatives, dibenzocycloheptadiene derivatives, dibenzodiazepine derivatives, dibenzoxepine derivatives, benzodiazepines, indole derivatives, phenylethylamine derivatives and hypericin derivatives, with the active agent being selected from the group which comprises chlorpromazine, perphenazine, sulpiride, clozapine, risperidone, reserpine, lorazepam, mirtazapine, maprotiline, mianserin, tranylcypromine, moclobemide, oxitriptan, viloxazine, reboxetine, meprobamate, hydroxyzine, buspirone, caffeine, fenetylline, methylphenidate, prolintane, fenfluramine, meclofenoxate, nicergoline, piracetam, pyritinol, as well as pharmaceutically acceptable salts of these active agents.

In one particular embodiment, the antidepressant is a selective serotonin reuptake inhibitor (SSRI), such as fluoxetine or paroxetine.

Preferably, tranylcypromine, reboxetine, lorazepam, mirtazapine, fluoxetine and paroxetine are used as antidepressants.

To improve the physicochemical properties, for example reduce the brittleness or embrittlement, humectants, such as glycerine, propylene glycol, sorbitol, mannitol, polyethylene glycol, polyglycerol ester and the like, may be added to the film.

In a further embodiment, antioxidants, for example vitamin C (ascorbic acid), ascorbyl palmitate, vitamin E (tocopherol acetate), hydroxybenzoic acid derivatives, may be added to the wafer, in order to stabilise the film and the active agents. Furthermore, acidic and basic ion exchangers may be used as stabilisers.

In further embodiments, further ingredients such as dyes, pigments, taste flavourings, natural and/or synthetic flavouring substances, sweeteners, buffering systems, may be added to the film. In particular, the taste flavourings and flavouring substances can mask the often bad inherent taste or smell of the active agents and/or give the dosage form a pleasant taste, so that the patient's readiness to take the medication is considerably improved.

The addition of buffering systems on the one hand serves to stabilise the film and the active agents against outside influences and during storage. On the other hand, the pH of the dosage form can thereby be adjusted to a physiologically acceptable pH value, so that irritation of mucous membranes is avoided. By using a buffering system, it is also possible to improve the solubility of acidic or basic active agents in the matrix.

The dosage forms according to the invention are configured so as to be thin, for example in the form of a wafer. The thickness of the dosage form is preferably 0.1 to 5 mm, more preferably 0.5 to 1 mm. The lower limit for the thickness of the dosage form is about 50 μm. The surface area of the dosage form is between 0.09 cm² and 12 cm², preferably between 1 cm² and 8 cm², and more preferably between 3 cm² and 6 cm².

In a further embodiment, the wafers of the present invention contain a disintegrant or a wicking agent, for example a bicarbonate-acid mixture or an aerosil, being activated by contact with a liquid and accelerating the disintegration of the wafer after application thereof, and thereby also accelerating the release of active agent.

In one preferred embodiment, the wafer is present as a foam so that the release of active agent takes place even more rapidly because of the enlarged surface. In this embodiment, the cavities of the foam may contain one or more of the active agents in liquid form.

To improve the absorption of the active agents via the mucous membrane, permeation enhancers, such as substances from the groups of the fatty alcohols, fatty acids, polyoxyethylene fatty alcohol ethers, polyoxyethylene fatty acid esters, fatty alcohol esters and fatty acid esters, particularly sorbitan monolaurate or esters of long-chain fatty acids with methyl, ethyl or isopropyl alcohol, or esters of fatty alcohols with acetic acid or lactic acid, or substances such as DMSO (dimethyl sulfoxide) and oleic acid diethanolamine may likewise be incorporated in the film. The constituent amount of these substances is 0.1 to 25%-wt., preferably 1 to 10%-wt., in each case relative to the total weight of the active agent matrix.

Furthermore, the composition of the wafer may contain compounds that retard the release of active agent (e.g. microencapsulation).

In a further embodiment, the wafer has mucoadhesive properties, so that it adheres to the mucous membrane until it is completely dissolved.

In a preferred embodiment, at least one of the active agents is bound to an ion exchanger, so that the hydrophilic polymer disintegrates quickly in the oral cavity, whereas the release of active agent is retarded or occurs when the pH has changed, e.g. in the gastrointestinal tract. In this way, active agents having a different mechanism of action and absorption can be administered in one dosage form, that is, at least one of the released active agents is either absorbed at the site of application, for example via the mucous membrane, or it is transported farther and absorbed at another location.

The wafer may also be made up as a laminate with different layers, with the active agents being contained in discrete layers which are spatially separated and differ from each other in terms of their composition. In this way, the active agents can be released at different sites of action, but also with retardation if the disintegration times of the various layers of the wafer differ from each other.

Likewise, the active agents may be arranged within layers that disintegrate at different rates, so that the preparation as a whole shows a retardation effect.

In a further embodiment, one of the outer layers may be mucoadhesive, to promote the adherence of the dosage form on the mucous membrane and to facilitate the active agent absorption via the mucous membrane by establishing direct contact.

The disintegration of the inventive dosage form in an aqueous medium preferably takes place in the range from 1 second to 5 minutes, more preferably in a range from 5 seconds to 1 minute, and most preferably in the range from 10 seconds to 30 seconds.

The dosage forms according to the invention are advantageously suitable for administering medicaments in the oral cavity or for rectal, vaginal or intranasal administration. They can be used both in human medicine and in veterinary medicine.

The present invention is furthermore directed to the use of one of the active agent combinations of the invention for the production of an oral dosage form for pain therapy, said dosage form preferably being formulated as a wafer.

Furthermore, the present invention relates to a method for the therapeutic treatment of a person suffering from pain, wherein the administration of an above-described active agent combination is carried out by means of an orally applicable dosage form with at least partial transmucosal absorption of the active agents.

Finally, the present invention also relates to a method for the production of a sheet-like dosage form, comprising the following steps:

preparing a solution containing at least one polymer and at least two of the above-described active agents;

spread-coating the solution on a coating substrate, and

solidifying the spread-coated solution by drying and withdrawing the solvent.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

We claim:

1. A sheet-like pharmaceutical preparation (dosage form) for pain therapy, based on hydrophilic polymers, which rapidly disintegrates upon contact with moisture and which releases at least one active agent in a body orifice or body cavity, wherein said dosage form contains an active agent combination of an opioid and a second substance selected from the group consisting of non-steroidal antirheumatics (NSAR's) and antidepressants.

2. The pharmaceutical preparation according to claim 1, wherein the opioid is selected from the group consisting of morphine, buprenorphine, hydromorphone, nalbuphine, fentanyl, sufentanil, alfentanil, remifentanil, tilidine, oxycodone, pethidine, levomethadone, piritramide and pentazocine, as well as pharmacologically acceptable salts of said compounds and combinations of two or more of the aforementioned compounds.

3. The pharmaceutical preparation according to claim 1, wherein the NSAR is selected from the group consisting of acetylsalicylic acid, phenylbutazone, oxyphenbutazone, acemetacine, diclofenac, indomethacin, lonazolac, mefenamic acid, niflumic acid, ibuprofen, ketoprofen, naproxen, tiaprofenic acid, piroxicam, tenoxicam and meloxicam, as well as pharmacologically acceptable salts and combinations of said active agents.

4. The pharmaceutical preparation according to claim 1, wherein the antidepressant is selected from the group consisting of phenothiazines, azaphenothiazines, thioxanthenes, butyrophenones, diphenylbutyl piperidines, iminodibenzyl derivatives, iminostilbene derivatives, dibenzocycloheptadiene derivatives, dibenzodiazepine derivatives, dibenzoxepine derivatives, benzodiazepines, indole derivatives, phenylethylamine derivatives and hypericin derivatives, as well as pharmaceutically acceptable salts or derivatives of said compounds.

5. The pharmaceutical preparation according to claim 1, wherein the active agent of the antidepressant is selected from the group consisting of chlorpromazine, perphenazine, sulpiride, clozapine, risperidone, reserpine, lorazepam, mirtazapine, maprotiline, mianserin, tranylcypromine, moclobemide, oxitriptan, viloxazine, reboxetine, meprobamate, hydroxyzine, buspirone, caffeine, fenetylline, methylphenidate, prolintane, fenfluramine, meclofenoxate, nicergoline, piracetam and pyritinol, as well as pharmacologically acceptable salts of said active agents.

6. The pharmaceutical preparation according to claim 5, wherein the antidepressant is selected from the group consisting of selective serotonin reuptake inhibitors (SSRI).

7. The pharmaceutical preparation according to claim 1, wherein the hydrophilic polymer is selected from the group consisting of dextran, polysaccharides, inclusive of starch and starch derivatives, cellulose derivatives, polyvinyl alcohols, polyethylene glycols, polyacrylic acids, polyacrylates, polyvinylpyrrolidones, alginates, pectins, gelatine, alginic acid, collagen, chitosan, arabinogalactan, galactomannan, agar-agar, agarose, carrageenan natural gums, tragacanth, highly dispersed silicon dioxide, bentonite, as well as derivatives of the aforementioned hydrophilic polymers or combinations of two or more of said polymers.

8. The pharmaceutical preparation according to claim 1, wherein the polymer film comprises a polyvinyl, alcohol-polyethylene glycol graft copolymer.

9. The pharmaceutical preparation according to claim 1, wherein the preparation further comprises a humectant selected from the group consisting of glycerine, propylene glycol, sorbitol, mannitol, polyethylene glycol and polyglycerol ester.

10. The pharmaceutical preparation according to claim 1, wherein the preparation further comprises an antioxidant selected from the group consisting of vitamin C (ascorbic acid), ascorbyl palmitate, vitamin E (tocopherol acetate) and hydroxybenzoic acid derivatives.

11. The pharmaceutical preparation according to claim 1, wherein the active agent of the preparation is bound to an acidic or basic ion exchanger for taste masking.

12. The pharmaceutical preparation according to claim 1, wherein the preparation contains dyes and/or pigments.

13. The pharmaceutical preparation according to claim 1, wherein the preparation further comprises at least one of natural and synthetic flavouring substances.

14. The pharmaceutical preparation according to claim 1, wherein the preparation further comprises a disintegrant or a wicking agent.

15. The pharmaceutical preparation according to claim 1, further comprising a buffer system for adjusting the pH value of the preparation.

16. The pharmaceutical preparation according to claim 1, wherein the hydrophilic polymer disintegrates within less than 5 minutes after application in the oral cavity of a user of the pharmaceutical preparation.

17. The pharmaceutical preparation according to claim 1, wherein the hydrophilic polymer disintegrates quickly in the oral cavity of a user of the pharmaceutical preparation whereas the active agent remains bound to an ion exchanger, said ion exchanger releasing said active agent only in the gastrointestinal tract.

18. The pharmaceutical preparation according to claim 1, wherein the active agents are contained in discrete layers which are spatially separated from each another and which differ from each other in terms of their respective composition.

19. The pharmaceutical preparation according to claim 1, wherein the preparation is present as a foam having cavities and at least one of the active agents is present in liquid form within the cavities of said foam.

20. The pharmaceutical preparation according to claim 1, wherein said preparation comprises a combination of two active agents.

21. Use of a dosage form according to claim 1, for rectal, vaginal or intra-nasal administration of pharmaceutical active agents to humans or animals.

22. Use of an active agent combination of opioid and NSAR, for the production of an oral dosage form according to claim 1 for treating pain disorders.

23. Use of an active agent combination of opioid and antidepressant for the production of an oral dosage form according to claim 1, for treating pain disorders.

24. Use according to claim 21, wherein the pharmaceutical product is formulated as a wafer.

25. A method for therapeutic pain treatment of a person suffering from chronic pain, comprising the step of orally administering to the person an active agent combination of opioid and NSAR in the form of an orally applicable dosage form with transmucosal absorption.

26. A method for therapeutic pain treatment of a person suffering from chronic pain, comprising the step of orally administering to the person an active agent combination of opioid and antidepressant in the form of an orally applicable dosage form with transmucosal absorption.

27. A method for producing a sheet-like dosage form for pain therapy, based on hydrophilic polymers, which rapidly disintegrates upon contact with moisture and which releases at least one active agent in a body orifice or body cavity, wherein said dosage form contains an active agent combination of an opioid and a second substance selected from the group consisting of non-steroidal antirheumatics (NSAR's) and antidepressants, said method comprising the steps of:
preparing a solution containing at least one polymer and at least two active agents;
spread-coating the solution on a coating substrate, and;
solidifying the spread-coated solution by drying and withdrawing the solvent.

28. The pharmaceutical preparation according to claim 6, wherein said selective serotonin reuptake inhibitors (SSRI) is selected from the group consisting of fluoxetine and paroxetine.

29. The pharmaceutical preparation according to claim 7, wherein said cellulose derivatives are selected from the group consisting of carboxymethyl cellulose, ethyl cellulose or propyl cellulose, hydroxypropylmethyl cellulose, hydroxypropyl cellulose, sodium carboxymethyl cellulose, methyl cellulose, hydroxyethyl cellulose and hydroxypropylethyl cellulose.

30. The pharmaceutical preparation according to claim 16, wherein the hydrophilic polymer disintegrates within less than 3 minutes after application in the oral cavity.

31. The pharmaceutical preparation according to claim 30, wherein the hydrophilic polymer disintegrates within less than 1 minute after application in the oral cavity.

32. The pharmaceutical preparation according to claim 31, wherein the hydrophilic polymer disintegrates within less than 30 seconds, after application in the oral cavity.

33. The pharmaceutical preparation according to claim 20, wherein said preparation comprising a combination of two active agents comprises a combination selected from the group consisting of a) a combination of an opioid and an NSAR and b) a combination of an opioid and an antidepressant.
